(19) United States
(12) Patent Application Publication
(10) Pub. No.: US 2021/0219838 A1
(43) Pub. Date: Jul. 22, 2021

(54) System and Method for Detection and Quantification of Impairment Due to Cannabis Use

(71) Applicant: SYNCTHINK INC., Palo Alto, CA (US)

(72) Inventors: Daniel J. Beeler, Somerville, MA (US); Jonathan R. Allen, San Francisco, CA (US)

(21) Appl. No.: 17/150,906

(22) Filed: Jan. 15, 2021

Related U.S. Application Data
(60) Provisional application No. 62/964,048, filed on Jan. 21, 2020.

(51) Int. Cl. A61B 3/113; A61B 5/16; A61B 5/00

(52) U.S. Cl. CPC A61B 3/113; A61B 5/162; A61B 5/4088; A61B 5/163

(57) ABSTRACT

A method of testing a subject for impairment includes presenting an object to the subject and measuring gaze positions of the subject. The method further includes generating, using the measured gaze positions, a plurality of values of a gaze metric. The method further includes generating a cognition metric for the subject based on one or more of the group consisting of: a measure of a skewness of a distribution of the plurality of values of the gaze metric; and a measure of kurtosis of the distribution of the plurality of values of the gaze metric. The method further includes determining whether the cognition metric is indicative of cognitive impairment; and generating a report, based at least in part on the determination of whether the cognition metric is indicative of cognitive impairment, indicating the presence or absence of cognitive impairment.

### RELATED AND PRIORITY APPLICATIONS

This application claims priority to U.S. Provisional Application No. 62/964,048, filed Jan. 21, 2020, which is hereby incorporated by reference in its entirety.

This application is also related to U.S. patent application Ser. No. 15/099,427, filed Apr. 14, 2016, which is hereby incorporated by reference in its entirety.

### TECHNICAL FIELD

The disclosed embodiments relate generally to systems and methods of testing a person's ability to track and anticipate visual stimuli, and more specifically, to a method and system for detecting and generating metrics corresponding to Cannabis impairment in a person's visual tracking of a smoothly moving object.

### BACKGROUND

Pairing an action with anticipation of a sensory event is a form of attention that is crucial for an organism's interaction with the external world. The accurate pairing of sensation and action is dependent on timing and is called sensory-motor timing, one aspect of which is anticipatory timing. Anticipatory timing is essential to successful everyday living, not only for actions but also for thinking. Thinking or cognition can be viewed as an abstract motor function and therefore also requires accurate sensory-cognitive timing. Sensory-motor timing is the timing related to the sensory and motor coordination of an organism when interacting with the external world. Anticipatory timing is usually a component of sensory-motor timing and is literally the ability to predict sensory information before the initiating stimulus.

Anticipatory timing is essential for reducing reaction times and improving both movement and thought performance. Anticipatory timing only applies to predictable sensory-motor or sensory-thought timed coupling. The sensory modality (e.g., visual, auditory etc.), the location, and the time interval between stimuli, must all be predictable (i.e., constant, or consistent with a predictable pattern) to enable anticipatory movement or thought.

Without reasonably accurate anticipatory timing, a person cannot catch a ball, know when to step out of the way of a moving object (e.g., negotiate a swinging door), get on an escalator, comprehend speech, concentrate on mental tasks or handle any of a large number of everyday tasks and challenges. This capacity for anticipatory timing can become impaired with sleep deprivation, aging, alcohol, drugs, hypoxia, infection, clinical neurological conditions including but not limited to Attention Deficit Hyperactivity Disorder (ADHD), schizophrenia, autism and brain trauma (e.g., a concussion).

Marijuana (“Cannabis”) use has become legalized both for medical and recreational use in an increasing number of jurisdictions. Along with the increased use coming from legalization, the number of injuries and fatalities due to impaired usage of motor vehicles has also increased. With respect to Cannabis impairment, various drug test methodologies are used in medicine, sports, and law. Past Cannabis use is detectable through measurements of metabolic byproducts of tetrahydrocannabinol (THC) in urine, hair, and saliva (e.g., byproducts such as 11-nor-delta9-tetrahydrocannabinol-9-carboxylic acid (delta9-THC—COOH)). Unlike alcohol, however, for which impairment can be reasonably measured using a breathalyzer (and confirmed with a blood alcohol content measurement), valid detection for Cannabis is time-consuming, and existing tests cannot objectively determine a degree of impairment. The lack of suitable tests and agreed-upon intoxication levels is an issue in the legality of cannabis, especially regarding intoxicated driving and readiness to perform tasks (e.g., work-related tasks) that may impact the safety, wellbeing or security of the subject and others or that may impact the reliability of the results produced when performing those tasks.

### SUMMARY

Accordingly, there is a need for objective and quantifiable measurements of Cannabis impairment. A technical solution to this problem is provided, in accordance with some embodiments, based on an observation that Cannabis impairment can be detected and quantified through analysis of higher-order signals (e.g., skew and kurtosis) in gaze position while a subject tracks a stationary or smoothly-moving object (e.g., a predictable object).

To that end, in accordance with some embodiments, a method, system, and computer-readable storage medium are proposed for detecting cognitive impairment, and in particular detecting cognitive impairment resulting from Cannabis use. In accordance method is performed at a system having a computer system and a measurement apparatus to measure gaze positions of a respective eye of the subject. The computer system has one or more processors and memory storing one or more programs for execution by the one or more processors. The method further includes presenting an object to the subject. The method further includes, while presenting the object to the subject, measuring, using the measurement apparatus, the gaze positions of the respective eye of the subject. The method further includes generating, using the measured gaze positions of the respective eye, a plurality of values of a gaze metric. The method further includes generating a cognition metric for the subject based on one or more of the group consisting of: a measure of a skewness of a distribution of the plurality of values of the gaze metric; and a measure of kurtosis of the distribution of the plurality of values of the gaze metric. The method further includes determining whether the cognition metric is indicative of cognitive impairment and generating a report, based at least in part on the determination of whether the cognition metric is indicative of cognitive impairment, indicating the presence or absence of cognitive impairment.

Further, in accordance with some embodiments, a system of testing a subject for impairment includes a measurement apparatus to measure the subject's gaze position; a display; one or more processors; memory storing one or more programs. The one or more programs include instructions to perform any of the methods described herein.

Further, in accordance with some embodiments, a non-transitory computer readable storage medium stores one or more programs. The one or more programs comprise instructions that when executed by one or more processors of a computer system operatively coupled to a display and a measurement apparatus to measure a subject's gaze position and cause a system that includes the computer system, display and measurement apparatus to perform any of the methods described herein.

### BRIEF DESCRIPTION OF THE DRAWINGS

FIG. 1 is a block diagram illustrating a system for measuring a subject's ability to visually track a smoothly moving object in accordance with some embodiments.

FIG. 2 is a conceptual block diagram illustrating a cognition timing diagnosis and training system in accordance with some embodiments.

FIG. 3 is a detailed block diagram illustrating a cognition timing diagnosis and training system in accordance with some embodiments.

FIGS. 4A-4F illustrate a smoothly moving object, moving over a tracking path, in accordance with some embodiments.

FIG. 5A shows eye movements obtained from a control subject (e.g., someone who is not impaired), following a target moving along a circular path.

FIG. 5B shows the eye movements of FIG. 5A, plotted in a target-based reference frame in which the target, actually moving clockwise, is fixed at the 12 o'clock position.

FIG. 5C shows eye movements obtained from a subject with Cannabis impairment, following a target moving along a circular path.

FIG. 5D shows the eye movements of FIG. 5C, plotted in a target-based reference frame in which the target, actually moving clockwise, is fixed at the 12 o'clock position.

FIG. 6A illustrates a distribution of tangential tracking errors for a control (unimpaired) subject and a subject with Cannabis impairment.

FIG. 6B illustrates a distribution of radial tracking errors for a control (unimpaired) subject and a subject with Cannabis impairment.

FIGS. 7A-7B illustrate a flowchart of a method of testing a subject for cognitive impairment (e.g., Cannabis impairment), in accordance with some embodiments.

Like reference numerals refer to corresponding parts throughout the several views of the drawings.

### DETAILED DESCRIPTION OF EMBODIMENTS

While physical movement by a subject can be measured directly, cognition, which is thinking performance, must be inferred. However, since cognition and motor timing are linked through overlapping neural networks, diagnosis and therapy can be performed for anticipatory timing difficulties in the motor and cognitive domains using motor reaction times and accuracy. In particular, both the timing and accuracy of a subject's movements can be measured. As discussed below, these measurements can be used for both diagnosis and therapeutic indications.

Anticipatory cognition and movement timing are controlled by essentially the same brain circuits. Variability or a deficit in anticipatory timing produces imprecise movements and is indicative of disrupted thinking, such as difficulty in concentration, memory recall, and carrying out both basic and complex cognitive tasks. Such variability and/or deficits leads to longer periods of time to successfully complete tasks and also leads to more inaccuracy in the performance of such tasks. Accordingly, in some embodiments, such variability is measured to determine whether a person suffers impaired anticipatory timing. In some embodiments, a sequence of stimuli is used in combination with a feedback mechanism to train a person to improve anticipatory timing.

As discussed in more detail below, in some embodiments, sequenced stimuli presented to a subject are or include predictable stimuli, for example, a smoothly and cyclically moving visual object. In some embodiments, non-predictable stimuli are presented to a subject before the predictable stimuli. The subject's responses to visual stimuli are typically visual, and in some of such embodiments, the subject's responses are measured by tracking eye movement. In some embodiments, a frontal brain electroencephalographic (EEG) signal (e.g., the “contingent negative variation” signal) is measured during the period in which a subject responds to the stimuli presented to the subject. The amplitude of the EEG signal is proportional to the degree of anticipation and will be disrupted when there are anticipatory timing deficits.

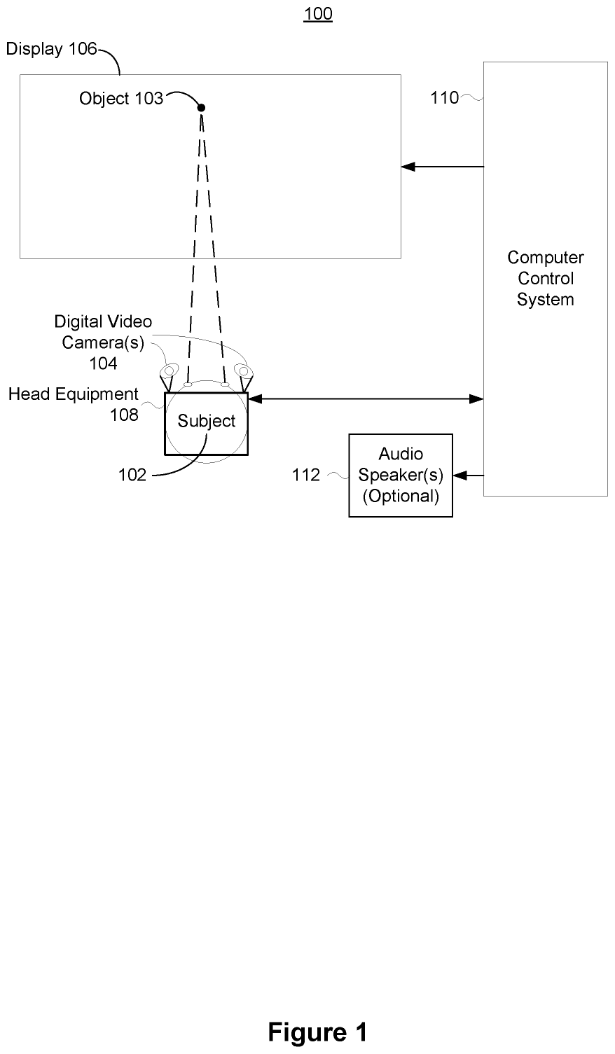

FIG. 1 illustrates a system 100 for measuring a subject's ability to visually track a moving object having predictable movements, typically a repeatedly performed sequence of movement, in accordance with some embodiments. More specifically, system 100 is configured to measure a subject's ability to visually track a smoothly moving object, in accordance with some embodiments. In some embodiments, the smoothly moving object is an object that moves along a continuous path (e.g., a circular path, or oval or elliptical path, rectangular path, or other continuous path) with a rate of movement that is constant, or a rate of movement that is the same at each location along the path each time the object moves through the path, or a rate of movement that follows a regular pattern discernable by ordinary human observers. However, in some other embodiments, movement of the object is continuous over a portion of the object's path, with a rate of movement that is constant or smoothly varying, and is non-continuous over another portion of the object's path (e.g., the object skips over certain portions of the path). In both types of embodiments, however, movement of the object is predictable by normal subjects due to the object's repeated movement over the same path.

In some embodiments, subject 102 is shown smoothly moving object 103 (e.g., a dot or ball moving at a constant speed), following a path (e.g., a circular or oval path) on display 106 (e.g., a screen). Measurement apparatus, such as digital video cameras 104, are focused on subject 102's eyes so that eye positions (and, in some embodiments, eye movements) of subject 102 are recorded. In accordance with some embodiments, digital video cameras 104 are mounted on subject 102's head by head equipment 108 (e.g., a headband or headset). Various mechanisms are, optionally, used to stabilize subject 102's head, for instance to keep the distance between subject 102 and display 106 fixed, and to also keep the orientation of subject 102's head fixed as well. In one embodiment, the distance between subject 102 and display 106 is kept fixed at approximately 40 cm. In some implementations, head equipment 108 includes the head equipment and apparatuses described in U.S. Patent Publication 2010/0204628 A1, which is incorporated by reference in its entirety. In some embodiments, the display 106, digital video cameras 104, and head equipment 108 are incorporated into a portable headset, configured to be worn by the subject while the subject's ability to track the smoothly moving object is measured. In some embodiments, head equipment 108 includes the headset described in U.S. Pat. No. 9,004,687, which is incorporated by reference in its entirety.

Display 106 is, optionally, a computer monitor, projector screen, or other display device. Display 106 and digital video cameras 104 are coupled to computer control system 110. In some embodiments, computer control system 110 controls the display of object 103 and any other patterns or objects or information displayed on display 106, and also receives and analyzes the eye position information received from the digital video cameras 104.

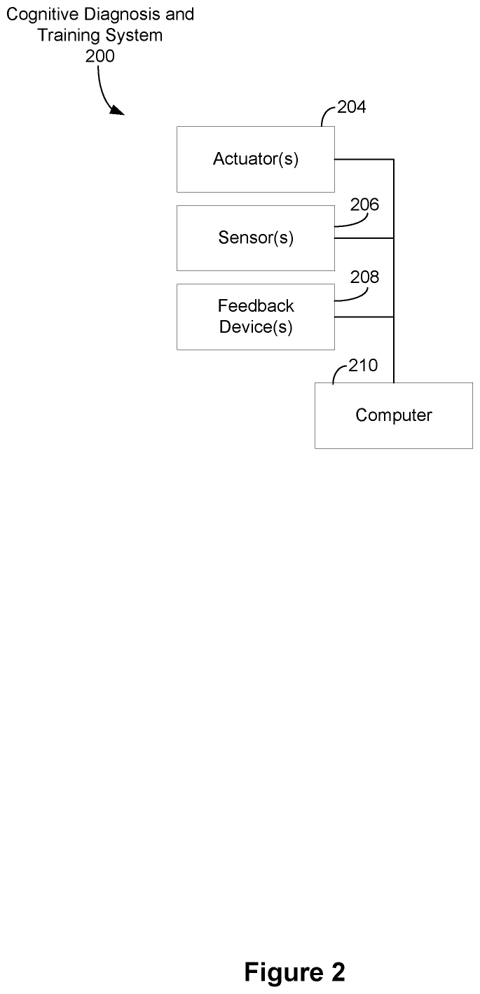

FIG. 2 illustrates a conceptual block diagram of a cognition diagnosis system 100, or a cognition and training system 200, in accordance with some embodiments. System 200 includes computer 210 (e.g., computer control system 110, FIG. 1) coupled to one or more actuators 204, and one or more sensors 206. In some embodiments, system 200 includes one or more feedback devices 208 (e.g., when system 200 is configured for use as a cognitive timing training system). In some embodiments, feedback is provided to the subject via the actuators 204. In some embodiments, actuators 204 include a display device (e.g., display 106, FIG. 1) for presenting visual stimuli to a subject. More generally, in some embodiments, actuators 204 include one or more of the following: a display device for presenting visual stimuli to a subject, audio speakers (e.g., audio speakers 112, FIG. 1) for presenting audio stimuli, a combination of the aforementioned, or one or more other devices for producing or presenting sequences of stimuli to a subject. In some embodiments, sensors 206, are, optionally, mechanical, electrical, electromechanical, auditory (e.g., microphone), or visual sensors (e.g., a digital video camera), or other type of sensors (e.g., a frontal brain electroencephalograph, sometimes called an EEG). The primary purpose of sensors 206 is to detect responses by a subject (e.g., subject 102 in FIG. 1) to sequences of stimuli presented by actuators 204. Some types of sensors produce large amounts of raw data, only a small portion of which can be considered to be indicative of the subject's response. In such systems, computer 210 contains appropriate filters and/or software procedures for analyzing the raw data so as to extract “sensor signals” indicative of the subject's response to the stimuli. In embodiments in which sensors 206 include an electroencephalograph (EEG), the relevant sensor signals from the EEG may be a particular component of the signals produced by the EEG, such as the contingent negative variation (CNV) signal or the readiness potential signal.

Feedback devices 208 are, optionally, any device appropriate for providing feedback to the subject (e.g., subject 102 in FIG. 1). In some embodiments, feedback devices 208 provide real time performance information to the subject corresponding to measurement results, which enables the subject to try to improve his/her anticipatory timing performance. In some embodiments, the performance information provides positive feedback to the subject when the subject's responses (e.g., to sequences of stimuli) are within a normal range of values. In some embodiments, the one or more feedback devices 208 may activate the one or more actuators 204 in response to positive performance from the subject, such as by changing the color of the visual stimuli or changing the pitch or other characteristics of the audio stimuli.

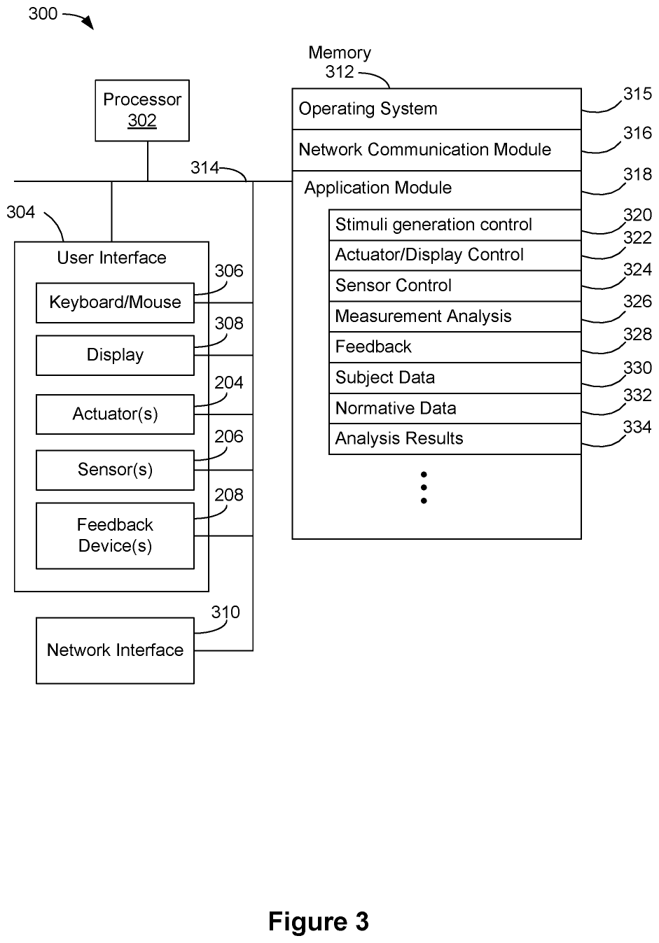

FIG. 3 is a block diagram of a cognition timing diagnosis and training (or remediation) system 300 in accordance with some embodiments. System 300 includes one or more processors 302 (e.g., CPUs), user interface 304, memory 312, and one or more communication buses 314 for interconnecting these components. In some embodiments, system 300 includes one or more network or other communications interfaces 310, such as a network interface for conveying testing or training results to another system or device. User interface 304 includes at least one or more actuators 204 and one or more sensors 206, and, in some embodiments, also includes one or more feedback devices 208. In some embodiments, actuator(s) 204 and sensor(s) 206 are implemented in a headset, while the remaining elements are implemented in a computer system coupled (e.g., by a wired or wireless connection) to the headset. In some embodiments, the user interface 304 includes computer interface devices such as keyboard/mouse 306 and display 308.

In some implementations, memory 312 includes a non-transitory computer readable medium, such as high-speed random access memory and/or non-volatile memory (e.g., one or more magnetic disk storage devices, one or more flash memory devices, one or more optical storage devices, and/or other non-volatile solid-state memory devices). In some implementations, memory 312 includes mass storage that is remotely located from processing unit(s) 302. In some embodiments, memory 312 stores an operating system 315 (e.g., Microsoft Windows, Linux or Unix), an application module 318, and network communication module 316.

In some embodiments, application module 318 includes stimuli generation control module 320, actuator/display control module 322, sensor control module 324, measurement analysis module 326, and, optionally, feedback module 328. Stimuli generation control module 320 generates sequences of stimuli, as described elsewhere in this document. Actuator/display control module 322 produces or presents the sequences of stimuli to a subject. Sensor control module 324 receives sensor signals and, where appropriate, analyzes raw data in the sensor signals so as to extract sensor signals indicative of the subject's (e.g., subject 102 in FIG. 1) response to the stimuli. In some embodiments, sensor control module 324 includes instructions for controlling operation of sensors 206. Measurement analysis module 326 analyzes the sensor signals to produce measurements and analyses, as discussed elsewhere in this document. Feedback module 328, if included, generates feedback signals for presentation to the subject via the one or more actuators or feedback devices.

In some embodiments, application module 318 further stores subject data 330, which includes the measurement data for a subject, and analysis results 334 and the like. In some embodiments, application module 318 stores normative data 332, which includes measurement data from one or more control groups of subjects, and optionally includes analysis results 334, and the like, based on the measurement data from the one or more control groups.

Still referring to FIG. 3, in some embodiments, sensors 206 include one or more digital video cameras focused on the subject's pupil (e.g., digital video cameras 104), operating at a picture update rate of 30 hertz or more. In some embodiments, the one or more digital video cameras are infrared cameras, while in other embodiments, the cameras operate in other portions of the electromagnetic spectrum. In some embodiments, the resulting video signal is analyzed by processor 302, under the control of measurement analysis module 326, to determine the screen positions, sometimes herein called gaze positions, where the subject focused, and the timing of when the subject focused at one or more predefined screen positions. For purposes of this discussion, the location of a subject's focus is the center of the subject's visual field. For example, using a picture update rate of 100 hertz, during a predefined test period of N seconds (e.g., 30 seconds), N×100 gaze position measurements are obtained, or 3000 gaze position measurements in 30 seconds. In another example, using a picture update rate of 500 hertz, during a predefined test period of N seconds (e.g., 30 seconds), N×500 gaze position measurements are obtained, or 15,000 gaze position measurements in 30 seconds.

In some embodiments, not shown, the system shown in FIG. 3 is divided into two systems, one which tests a subject and collects data, and another which receives the collected data, analyzes the data and generates one or more corresponding reports.

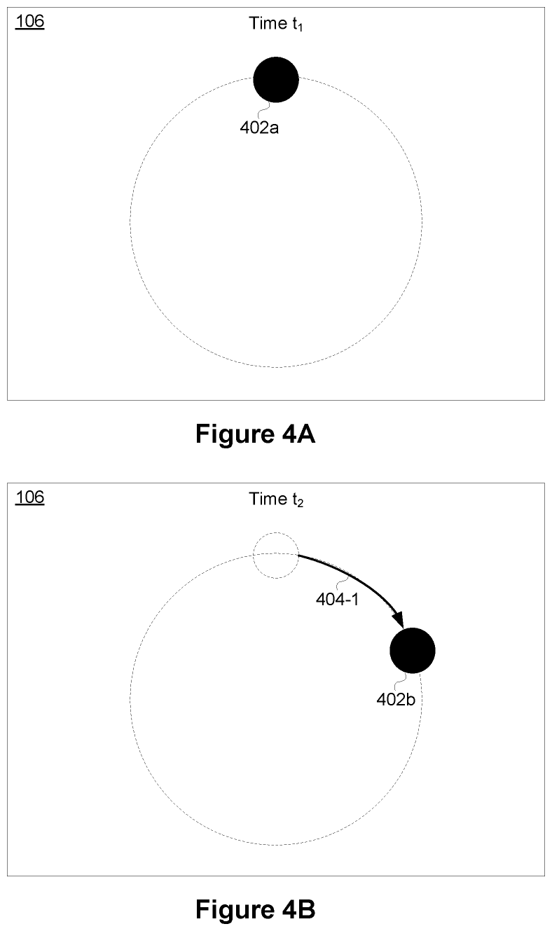

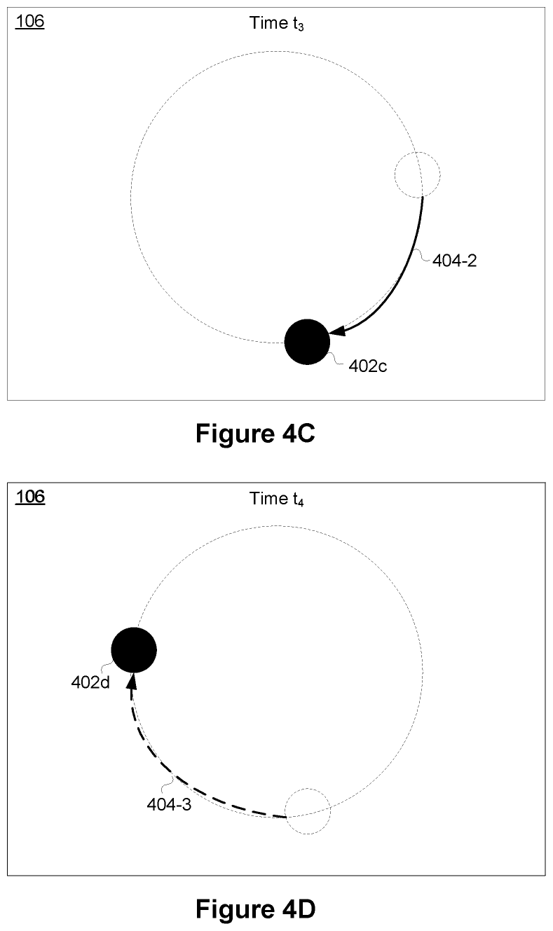

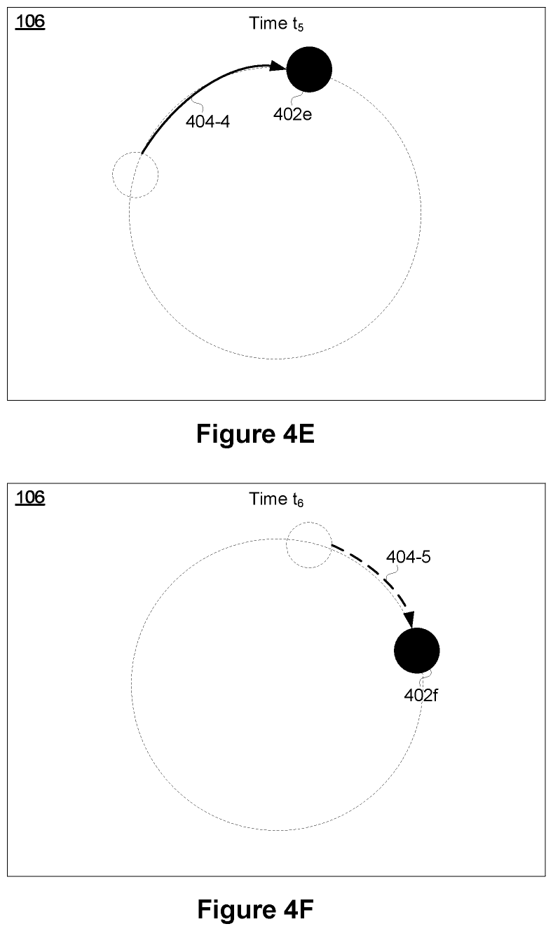

Ocular Pursuit. FIGS. 4A-4F illustrate a smoothly moving object, moving over a tracking path in accordance with some embodiments. FIG. 4A shows object 402 (e.g., a dot) at position 402a on display 106 (on the tracking path) at time t₁. FIG. 4B shows object 402 move along tracking path segment 404-1 to position 402b at time t₂. FIG. 4C shows object 402 move along tracking path segment 404-2 to position 402c at time t₃. FIG. 4D shows object 402 move along tracking path segment 404-3 to position 402d at time t₄. Tracking path segment 404-3 is shown as a dotted line to indicate that object 402 may or may not be displayed while moving from position 402c to position 402d (e.g., tracking path segment 404-3 represents a gap in tracking path 404 of object 402 when object 402 is not displayed on this path segment). FIG. 4E shows object 402 move along tracking path segment 404-4 to position 402e at time t₅. In some embodiments, position 402e is the same as position 402a and time t₅ represents the time it takes object 402 to complete one revolution (or orbit) along the tracking path. FIG. 4F shows object 402 moving along tracking path segment 404-5 to position 402f at time t₆. In some embodiments, position 402f is position 402b.

For purposes of this discussion the terms “normal subject” and “abnormal subject” are defined as follows. Normal subjects are healthy individuals without any known or reported impairments to brain function (including intoxication). Abnormal subjects are individuals suffering from impaired brain function with respect to sensory-motor or anticipatory timing.

Calibration. In some embodiments, in order to provide accurate and meaningful real time measurements of where the subject is looking at any one point in time, the eye position measurements (e.g., produced via digital video cameras 104) are calibrated by having the subject focus on a number of points on a display (e.g., display 106) during a calibration phase or process. For instance, in some embodiments, calibration may be based on nine points displayed on the display, including a center point, positioned at the center of the display locations to be used during testing of the subject, and eight points along the periphery of the display region to be used during testing of the subject. The subject is asked to focus on each of the calibration points, in sequence, while digital video cameras (e.g., digital video cameras 104) measure the pupil and/or eye position of the subject. The resulting measurements are then used by a computer control system (e.g., computer control system 110) to produce a mapping of eye position to screen location, so that the system can determine the position of the display at which the subject is looking at any point in time (referred to as “gaze positions”). In other embodiments, the number of points used for calibration may be more or less than nine points, and the positions of the calibration points may be distributed on the display in various ways.

In some implementations, the calibration process is performed each time a subject is to be tested, because small differences in head position relative to the cameras, and small differences in position relative to the display 106, can have a large impact on the measurements of eye position, which in turn can have a large impact of the “measurement” or determination of the display position at which the subject is looking. The calibration process can also be used to verify that the subject (e.g., subject 102) has a sufficient range of oculomotor movement to perform the test.

Ocular Pursuit to Assess Anticipatory Timing. In some embodiments, after calibration is completed, the subject is told to look at an object (e.g., a dot or ball) on the display and to do his/her best to maintain the object at the center of his/her vision as it moves. In some embodiments, stimuli generation control module 320 generates or controls generation of the moving object and determination of its tracking path, and actuator/display control module 322 produces or presents the sequences of stimuli to the subject. The displayed object is then smoothly moved over a path (e.g., a circular or elliptical path). In some embodiments, the rate of movement of the displayed object is constant for multiple orbits around the path. In various embodiments, the rate of movement of the displayed object, measured in terms of revolutions per second (i.e., hertz), is as low as 0.1 Hz and as high as 10 Hz. However, it has been found that the most useful measurements are obtained when the rate of movement of the displayed object is in the range of about 0.4 Hz to 1.0 Hz, and more generally when the rate of movement of the displayed object is in the range of about 0.2 Hz to 2.0 Hz. A rate of 0.4 Hz corresponds to 2.5 seconds for the displayed object to traverse the tracking path, while a rate of 1.0 Hz corresponds to 1.0 seconds for the displayed object to traverse the tracking path. Even normal, healthy subjects have been found to have trouble following a displayed object that traverses a tracking path at a repetition rate of more than about 2.0 Hz.

In some embodiments, the subject is asked to follow the moving object for eight to twenty circular orbits. For example, in some embodiments, the subject is asked to follow the moving object for twelve clockwise circular orbits having a rate of movement of 0.4 Hz, measured in terms of revolutions per second. Furthermore, in some embodiments, the subject is asked to follow the moving object for two or three sets of eight to twenty clockwise circular orbits, with a rest period between.

The angular amplitude of the moving object, as measured from the subject's eyes, is about 10 degrees in the horizontal and vertical directions. In other embodiments, the angular amplitude of the moving object, as measured from the subject's eyes, is 15 degrees or more. The eye movement of the subject, while following the moving displayed object, can be divided into horizontal and vertical components for analysis. Thus, in some embodiments, four sets of measurements are made of the subject's eye positions while performing smooth pursuit of a moving object: left eye horizontal position, left eye vertical position, right eye horizontal position, and right eye vertical position. Ideally, in such embodiments as those utilizing a circularly or elliptically moving visual object, if the subject perfectly tracked the moving object at all times, each of the four positions would vary sinusoidally over time. That is, a plot of each component (horizontal or vertical) of each eye's position over time would follow the function sin(ωt+θ), where sin( ) is the sine function, θ is an initial angular position, and w is the angular velocity of the moving object. In some embodiments, one or two sets of two dimensional measurements (based on the movement of one or two eyes of the subject) are used for analysis of the subject's ability to visually track a smoothly moving displayed object. In some embodiments, the sets of measurements are used to generate a tracking metric. In some embodiments, the sets of measurements are used to generate a disconjugacy metric by using a binocular coordination analysis.

In some embodiments, the subject is asked to focus on an object that is not moving, for a predefined test period of T seconds (e.g., 30 seconds, or any suitable test period having a duration of 15 to 60 seconds), measurements are made of how well the subject is able to maintain focus (e.g., the center of the subject's visual field) on the object during the test period, and an analysis, similar to other analyses described herein, is performed on those measurements. In some circumstances, this “non-moving object” test is performed on the subject in addition to the ocular pursuit test(s) described herein, and results from the analyses of measurements taken during both types of tests are used to evaluate the subjects cognitive function.

Ocular pursuit eye movement is an optimal movement to assess anticipatory timing in intentional attention (interaction) because it requires attention. Measurements of the subject's point of focus, defined here to be the center of the subject's visual field, while attempting to visually track a moving displayed object can be analyzed for binocular coordination so as to generate a disconjugacy metric. Furthermore, as discussed in more detail in published U.S. Patent Publication 2006/0270945 A1, which is incorporated by reference in its entirety, measurements of a subject's point of focus while attempting to visually track a moving displayed object can also be analyzed so as to provide one or more additional metrics, such as a tracking metric, a metric of attention, a metric of accuracy, a metric of variability, and so on.

In accordance with some implementations, for each block of N revolutions or orbits of the displayed object, the pictures taken by the cameras are converted into display locations (hereinafter called gaze positions), indicating where the subject was looking at each instant in time recorded by the cameras. In some embodiments, the gaze positions are compared with the actual displayed object positions to produce gaze errors.

Higher-Order Analysis of Gaze Measurements for Detecting Cannabis Impairment. Analysis of the results produced by testing of Cannabis-impaired subject using the smooth pursuit methodology described herein shows that such subject show deficits in synchronizing their gaze with the target motion during circular visual tracking, especially in higher-order moments (e.g., the third-order moment, referred to as “skew” and the fourth-order moment, referred to as “kurtosis”) of the distribution of gaze positions or gaze errors (e.g., examples of gaze metrics), while still engaged in predictive behavior per se. Note that, in general, a “moment” refers to a quantitative measure of the shape of a distribution of values. An Nth-order moment of a distribution of values (e.g., distribution of gaze metric values) is defined by any of the equivalent formulas below:

$${μ}_{N} = E [ {( \frac{X - {μ}_{1}}{σ} )}^{N} ] \\ {μ}_{N} = \frac{E [ {( X - {μ}_{1} )}^{N} ]}{( {E [ {( X - {μ}_{1} )}^{2} ]}^{N / 2} )} \\ {μ}_{N} = \frac{E [ {( X - {μ}_{1} )}^{N} ]}{{( E [ {( X - {μ}_{1} )}^{2} ] )}^{N / 2}} \\ {μ}_{N} = \frac{\frac{1}{n} {\sum }_{i = 1}^{n} {( {x}_{i} - \overline{x} )}^{N}}{{( \frac{1}{n - 1} {\sum }_{i = 1}^{n} {( {x}_{i} - \overline{x} )}^{2} )}^{N / 2}}$$

where N is the order of the moment; μ_{N} is the Nth-order moment; X is the set of values (e.g., samples or observations) for which individual values are labeled xᵢ; μ₁ and x̅ both represent the mean of the set of values (e.g., different notation for the mean); n is the number of values (e.g., number of samples in the observation); σ is the standard deviation of the set of values; and E the expectation operator. Note that the Equations above provide example forms of Nth-order moments, and that, in accordance with some embodiments, various other forms of Nth-order moments may be used instead. For example, in some embodiments, an Nth-order moment may be calculated without the denominators in the Equations above. Moreover, various other forms will be apparent to one of skill in the art, having had the benefit of this disclosure.

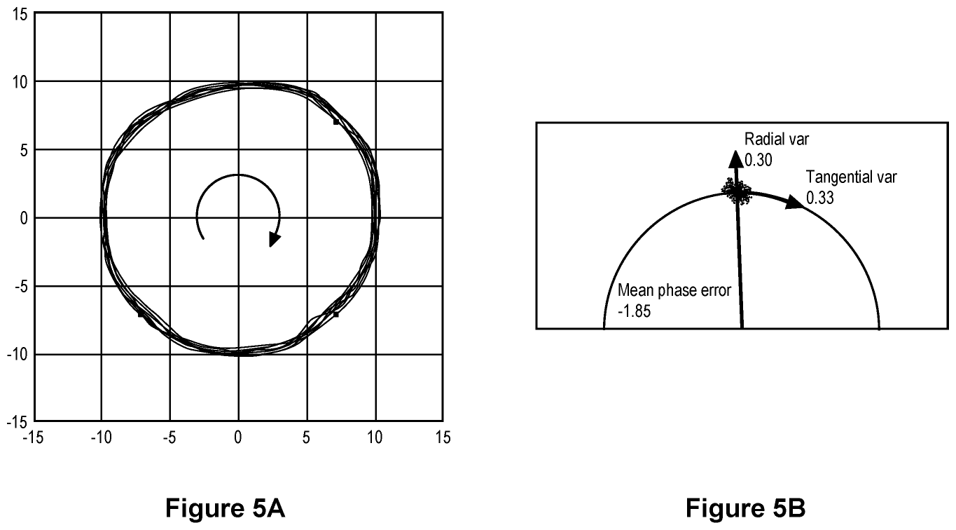

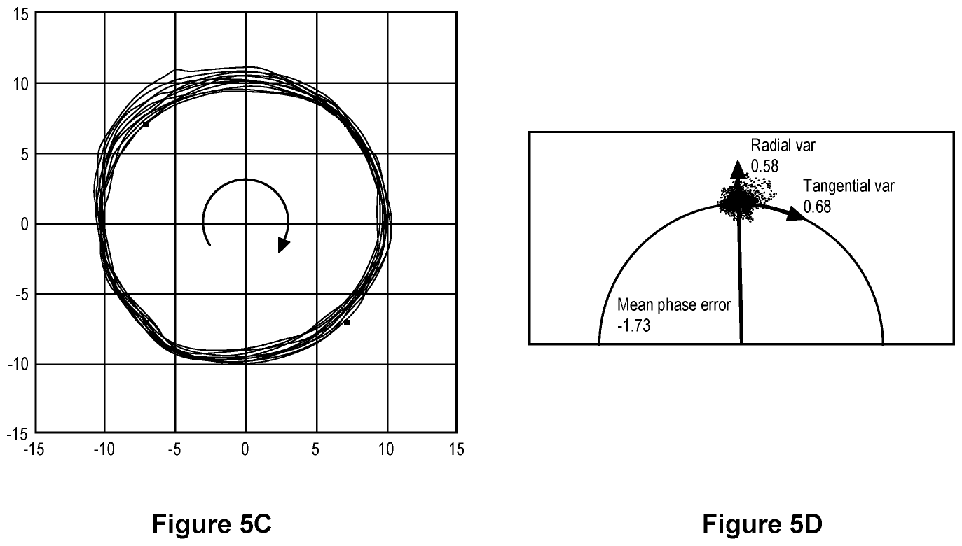

FIG. 5A depicts gaze positions (e.g., in arbitrary units of distance, such as millimeters) of measurement data from a control subject (e.g., someone who is not impaired), following a target moving along a circular path. FIG. 5B shows the same eye movements, plotted in a target-based reference frame in which the target, actually moving clockwise, is fixed at the 12 o'clock position. The units for tangential error are in degrees of visual angle, while the units for radial error are in the same arbitrary units of distance (e.g., millimeters). The standard deviation (or equivalently, variance (var)) values have the same units, respectively. FIGS. 5C-5D illustrate analogous data for a subject who is impaired from Cannabis.

In FIGS. 5B and 5D, a gaze point plotted at the 12 o'clock position on the circular path of the target is said to have a zero error. Thus, the data points shown in FIGS. 5B and 5D represent the subject's tracking errors over the course of the test period, with the tracking errors being shown in two dimensions, radial and tangential, relative to the circular trajectory of the target.

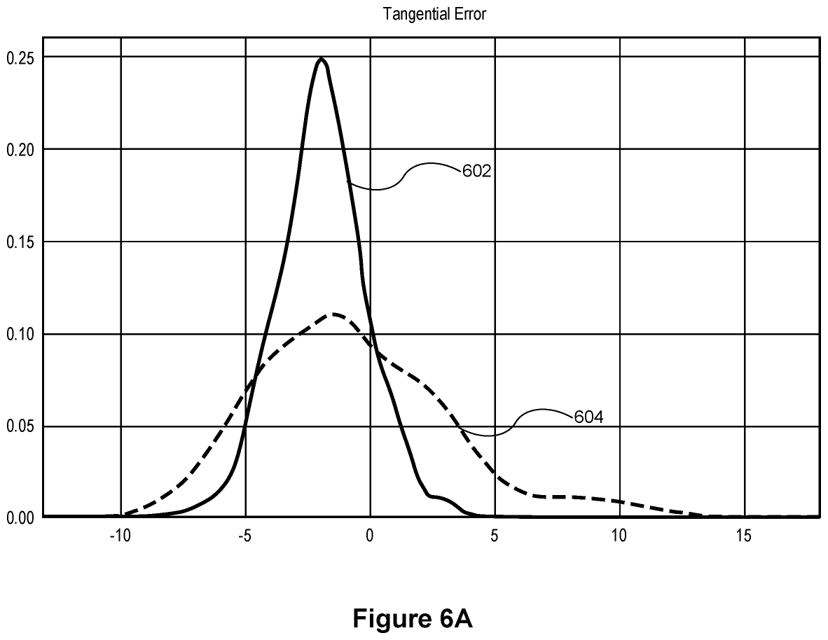

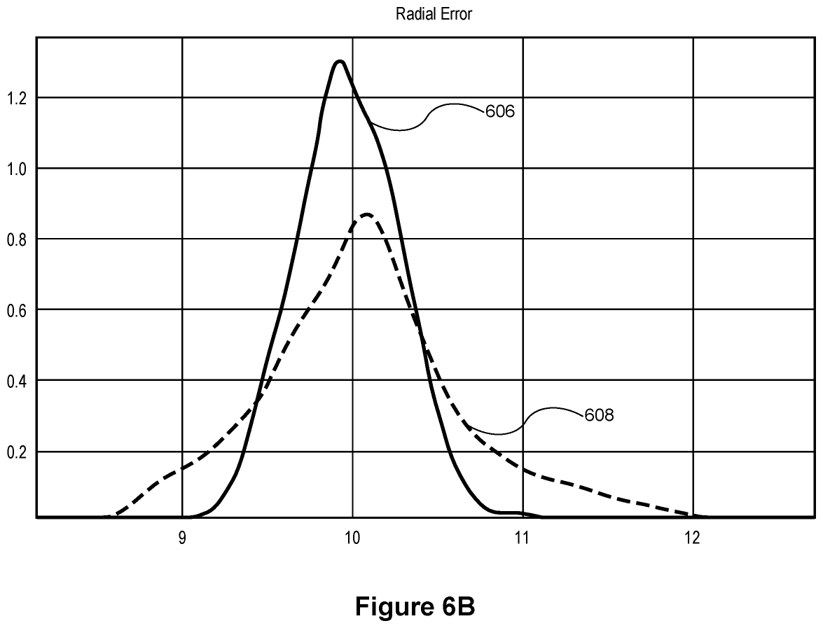

The error in the position between the subject's gaze position and the target position at a given instant of time can be decomposed into radial and tangential components defined relative to the target trajectory. The radial component represents the subject's spatial error in a direction orthogonal to the target trajectory, whereas the tangential component represents a combination of spatial and temporal errors in a direction parallel to the target trajectory. As an example, FIG. 6A illustrates a distribution (e.g., a probability density function) of tangential tracking errors for a control (unimpaired) subject (curve 602) and a subject with Cannabis impairment (curve 604), while FIG. 6B illustrates a distribution (e.g., a probability density function) of radial tracking errors for a control (unimpaired) subject (curve 606) and a subject with Cannabis impairment (curve 608). In FIGS. 6A-6B, the horizontal (abscissae) axis represents tracking error values (e.g., tangential and radial, respectively, in the units defined above) and vertical (ordinate) axes represent the frequency that measurements having the corresponding error values were observed during the testing period (note that the curves shown in FIGS. 6A-6B are interpolated to show a smoother curve).

As can be seen from a comparison of FIGS. 5A-5D and FIGS. 6A-6B the distributions of gaze measurements and gaze errors (e.g., examples of gaze metrics) are clearly different between the unimpaired and impaired subject. A non-impaired person exhibits an error distribution in the phase and radial aspects of tracking which has a measure of clear control and correction: the error is seen to have a large peak slightly behind the target with less frequent larger corrections which jump ahead of the target. When a subject has ingested Cannabis, they appear to lose a fair amount of control over the precision of their tracking (e.g., due to the effect Cannabis has on temporal aspects, as it binds cannabinoid receptors in the cerebellum). In fact, these differences can be used to determine whether the subject is experiencing Cannabis impairment (e.g., as opposed to other forms of impairment, such as impairment from traumatic brain injuries or disease processes) by analyzing higher-order moments of the distributions of gaze measurements and gaze errors.

More particularly, it has been observed that Cannabis impairment does not necessarily change the amount of gaze error that a subject demonstrates, but it consistently changes the type of that error: an individual who is impaired due to Cannabis will have an error distribution which is less controlled, and thus more Gaussian in nature. Physically, cannabinoid receptors are located in the cerebellum, which regulates timings. When cannabinoids bind to the receptors, the result is a dulling of reaction. The executive functions of the brain remain intact. The end result is that the subject tends not to react as quickly or to over-compensate when they do react, leading to an error distribution which is much more normal in its distribution. This distinction between sober behavior and impairment due to cannabis appears to be very strong and very consistent.

In fact, differences in higher-order moments between unimpaired and impaired individuals are strong enough to be able to determine whether a subject is impaired without a prior baseline for that subject or even necessarily correcting for additional demographics (e.g., age). This observation makes higher-order moments (e.g., skew and kurtosis) ideal candidates for assessing Cannabis impairment using eye tracking. Further, while it may be possible for the subject to intentionally alter certain lower-order metrics (e.g., gaze error) or other proxies for intoxication (e.g., blink rate), higher-order moments of gaze distributions are extremely difficult to intentionally alter. For example, it is unreasonable to expect that anyone could control the 3rd- and 4th-order moments of the distribution of their errors while impaired.

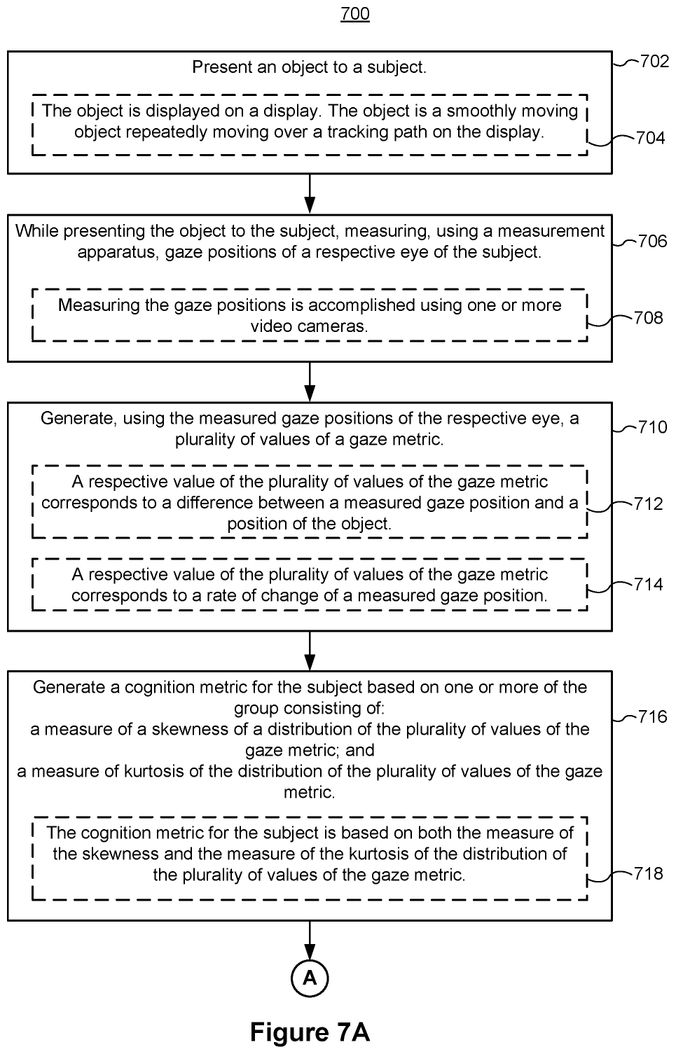

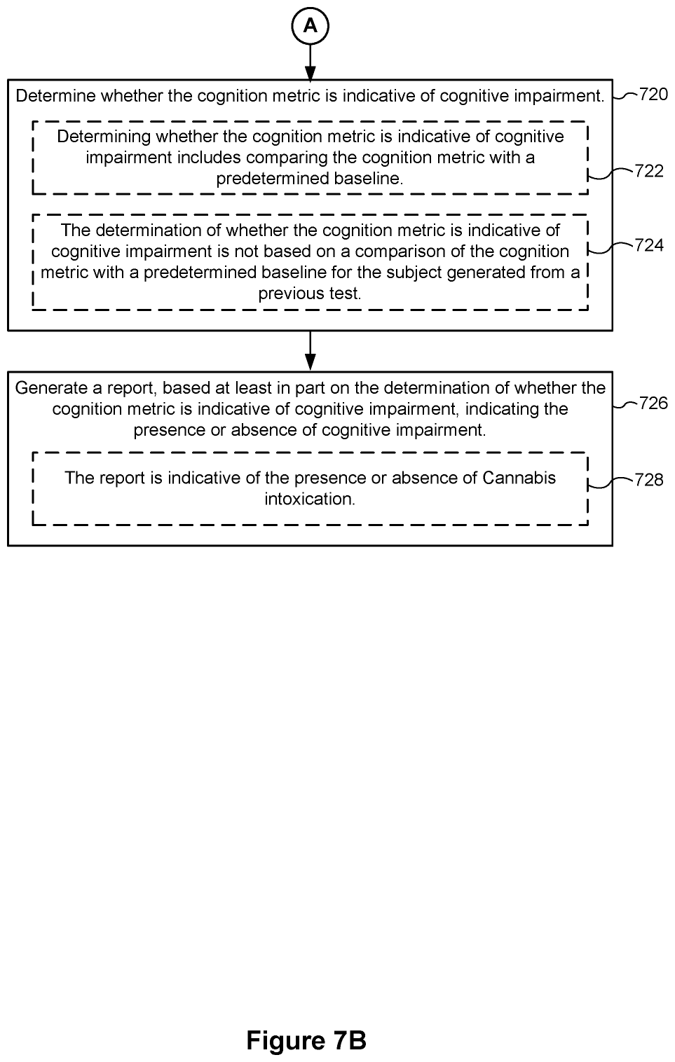

FIG. 7A-7B illustrate a flowchart of a method 700 of testing a subject for cognitive impairment (e.g., Cannabis impairment), in accordance with some embodiments.

In some embodiments, method 700 is performed by a system (e.g., system 100, FIG. 1) that includes a computer control system, a display, and a measurement apparatus to measure the subject's gaze positions (e.g., at least the gaze positions for a respective eye, such as the right eye or the left eye) over a period of time while viewing information displayed on the display. The computer system (e.g., computer system 300, FIG. 3) includes one or more processors and memory storing one or more programs for execution by the one or more processors. Under control of the one or more programs executed by the computer system, the method includes, presenting (702) an object to a subject (e.g., object 402, FIGS. 4A-4F). In some embodiments, the object is (704) displayed on a display. In some embodiments, the object is a smoothly moving object repeatedly moving over a tracking path on the display. In some embodiments, the object is presented to the subject during a predefined test period (e.g., a 30 second period). In some embodiments, sufficient data is obtained during the predefined test period to detect and quantify the presence or absence of Cannabis impairment.

Note that method 700 is described with respect to measurements of the subject's gaze position. However, in some embodiments, measurements of the subject's eye position, or other measurements based on measurements of the subject's eye position may be used.

Method 700 further includes, while presenting the object to the subject, measuring (706), using a measurement apparatus, gaze positions of a respective eye of the subject (e.g., the subject's left eye or the subject's right eye). In some embodiments, the measurement apparatus measures gaze positions for each of the subject's eyes. For example, as discussed above, the method may include making 100 to 500 measurements of gaze position per second, thereby generating a sequence of 3,000 to 15,000 gaze position measurements over a 30 second test period. In some embodiments, measuring the gaze positions is (708) accomplished using one or more video cameras.

Method 700 further includes generating (710), using the measured gaze positions (or eye positions) of the respective eye, a plurality of values of a gaze metric. In some embodiments, a respective value of the plurality of values of the gaze metric corresponds to (712) a difference between a measured gaze position and a position of the object (e.g., the gaze metric is a tracking error). In some embodiments, the gaze metric corresponds to a radial error component (e.g., an error in gaze position with respect to the target along a direction perpendicular to the movement of the target). In some embodiments, the gaze metric corresponds to a tangential error component (also called a phase error component) (e.g., an error in gaze position with respect to the target along a direction tangential to the movement of the target). In some embodiments, the gaze metric corresponds to the gaze position. In some embodiments, the gaze metric is a disconjugacy metric. In some embodiments, the gaze metric is independent of the position of the target.

In some embodiments, a respective value of the plurality of values of the gaze metric corresponds to (714) a rate of change of a measured gaze position (e.g., a speed of the gaze position or a tangential or radial component of the velocity of the gaze position).

Method 700 further includes generating (716) a cognition metric for the subject based on one or more of the group consisting of: a measure of a skewness of a distribution of the plurality of values of the gaze metric; and a measure of kurtosis of the distribution of the plurality of values of the gaze metric (e.g., the cognition metric is based on a third or higher moment of the distribution of the gaze metric). In some embodiments, the cognition metric for the subject is based (718) on both the measure of the skewness and the measure of the kurtosis of the distribution of the plurality of values of the gaze metric (e.g., a linear combination of the two).

In some embodiments, the cognition metric is further based on one or more of: a measure of blink loss (e.g., blink rate); a standard deviation of a distribution of a tangential component of differences between the measured gaze positions and the position of the object on the tracking path; and a standard deviation of a distribution of a phase error of differences between the measured gaze positions and the position of the object on the tracking path.

Method 700 further includes (720) determining whether the cognition metric is indicative of cognitive impairment. In some embodiments, determining (722) whether the cognition metric is indicative of cognitive impairment includes comparing the cognition metric with a predetermined baseline. In some embodiments the predetermined baseline is based on at least one of: a range from previous tests of a preselected group of unimpaired control subjects; and a range for the subject generated from one or more previous tests.

In some embodiments, the determination of whether the cognition metric is indicative of impairment is made without comparing the cognition metric with a predetermined baseline (e.g., a predetermined baseline for the subject or for a control group of people sharing one or more demographics with the subject). In some embodiments, the determination of whether the cognition metric is indicative of cognitive impairment is not based (724) on a comparison of the cognition metric with a predetermined baseline for the subject generated from a previous test.

In some embodiments, the cognition metric is a first cognition metric having a first false positive rate (or alternatively, a first specificity, also known as a true positive rate). Method 700 further includes generating a second cognition metric for the subject based, at least in part, on one or more of the group consisting of: a measure of a skewness of a distribution of the plurality of values of the gaze metric; and a measure of kurtosis of the distribution of the plurality of values of the gaze metric. In some embodiments, the group further consists of a measure of blink loss; a standard deviation of a distribution of a tangential component of differences between the measured gaze positions and the position of the object on the tracking path; and a standard deviation of a distribution of a phase error of differences between the measured gaze positions and the position of the object on the tracking path. In some embodiments, the first cognition metric is a first combination (e.g., linear combination) of any of the aforementioned factors and the second cognition metric is a second combination (e.g., linear combination) of any of the aforementioned factors.

The second cognition metric is distinct from the first cognition metric and has a second false positive rate that is lower than the first false positive rate (e.g., the second cognition metric has a second specificity that is higher than the first specificity of the first cognition metric). In some embodiments, the first cognition metric has a first false negative rate (or, alternatively, a first sensitivity, also known as a true negative rate) that is higher than a second false negative rate of the second cognition metric (e.g., the first cognition metric has a first sensitivity that is higher than a second sensitivity of the second cognition metric).

Method 700 further includes generating (726) a report, based at least in part on the determination of whether the cognition metric (or metrics) is indicative of cognitive impairment, indicating the presence or absence of cognitive impairment. In some embodiments, the report is indicative of the presence or absence of Cannabis intoxication (e.g., is specific to Cannabis intoxication as opposed to impairment from other, non-Cannabis causes). In some embodiments, a magnitude of the cognition metric corresponds to a degree of impairment of the subject. In some embodiments, generating the report includes separately presenting information corresponding to both the first cognition metric and the second cognition metric (e.g., presenting information indicating the presence or absence, and/or magnitude, of Cannabis impairment, together with a confidence score indicating a certainty of the presence or absence of impairment).

Providing both the indication of Cannabis impairment and the certainty of Cannabis impairment allows the information to be used for different types of purposes. For example, a higher degree of certainty may be needed by law enforcement to establish probable cause for arresting a subject for driving under the influence than is needed by an employer to prevent an employee from operating heavy machinery.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the invention and its practical applications, to thereby enable others skilled in the art to best utilize the invention and various embodiments with various modifications as are suited to the particular use contemplated.

It will be understood that, although the terms “first,” “second,” etc. may be used herein to describe various elements, these elements should not be limited by these terms. These terms are only used to distinguish one element from another. For example, a first sound detector could be termed a second sound detector, and, similarly, a second sound detector could be termed a first sound detector, without changing the meaning of the description, so long as all occurrences of the “first sound detector” are renamed consistently and all occurrences of the “second sound detector” are renamed consistently. The first sound detector and the second sound detector are both sound detectors, but they are not the same sound detector.

The terminology used herein is for the purpose of describing particular implementations only and is not intended to be limiting of the claims. As used in the description of the implementations and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” may be construed to mean “upon determining” or “upon a determination that” or “in response to determining” or “in accordance with a determination” or “upon detecting” or “in response to detecting” that the stated condition precedent is true, depending on the context.

What is claimed is:

1. A method of testing a subject for cognitive impairment, comprising:
at a system having a computer system and a measurement apparatus to measure gaze positions of a respective eye of the subject, the computer system having one or more processors and memory storing one or more programs for execution by the one or more processors, performing a set of operations including:
presenting an object to the subject;
while presenting the object to the subject, measuring, using the measurement apparatus, the gaze positions of the respective eye of the subject;
generating, using the measured gaze positions of the respective eye, a plurality of values of a gaze metric;
generating a cognition metric for the subject based on one or more of the group consisting of:
a measure of a skewness of a distribution of the plurality of values of the gaze metric; and
a measure of kurtosis of the distribution of the plurality of values of the gaze metric;
determining whether the cognition metric is indicative of cognitive impairment; and
generating a report, based at least in part on the determination of whether the cognition metric is indicative of cognitive impairment, indicating the presence or absence of cognitive impairment.

2. The method of claim 1, wherein a respective value of the plurality of values of the gaze metric corresponds to a difference between a measured gaze position and a position of the object.

3. The method of claim 1, wherein a respective value of the plurality of values of the gaze metric corresponds to a rate of change of a measured gaze position.

4. The method of claim 1, wherein the cognition metric for the subject is based on both the measure of the skewness and the measure of the kurtosis of the distribution of the plurality of values of the gaze metric.

5. The method of claim 1, wherein:
the object is displayed on a display; and
the object is a smoothly moving object repeatedly moving over a tracking path on the display.

6. The method of claim 5, further including determining a phase error of differences between the measured gaze positions and the position of the object on the tracking path,
wherein the measure of the skewness is based on the distribution of the phase error of the differences between the measured gaze positions and the position of the object on the tracking path.

7. The method of claim 5, further including determining a radial component of differences between the measured gaze positions and the position of the object on the tracking path,
wherein the measure of the skewness is based on the distribution of the radial component of the differences between the measured gaze positions and the position of the object on the tracking path.

8. The method of claim 5, further including determining a phase error of differences between the measured gaze positions and the position of the object on the tracking path,
wherein the measure of the kurtosis is based on the distribution of the phase error of the differences between the measured gaze positions and the position of the object on the tracking path.

9. The method of claim 5, wherein the cognition metric is further based on one or more of:
a measure of blink loss;
a standard deviation of a distribution of a tangential component of differences between the measured gaze positions and the position of the object on the tracking path; and
a standard deviation of a distribution of a phase error of differences between the measured gaze positions and the position of the object on the tracking path.

10. The method of claim 1, wherein determining whether the cognition metric is indicative of cognitive impairment includes comparing the cognition metric with a predetermined baseline.

11. The method of claim 10, wherein the predetermined baseline is based on at least one of:
a range from previous tests of a preselected group of unimpaired control subjects; and
a range for the subject generated from one or more previous tests.

12. The method of claim 1, wherein the determination of whether the cognition metric is indicative of cognitive impairment is not based on a comparison of the cognition metric with a predetermined baseline for the subject generated from a previous test.

13. The method of claim 1, wherein the report is indicative of the presence or absence of cannabis intoxication.

14. The method of claim 1, wherein measuring the gaze positions is accomplished using one or more video cameras.

15. The method of claim 1, wherein:
the cognition metric is a first cognition metric having a first false positive rate;
the method further includes:
generating a second cognition metric for the subject based on one or more of the group consisting of:
a measure of a skewness of a distribution of the plurality of values of the gaze metric; and
a measure of kurtosis of the distribution of the plurality of values of the gaze metric; and
the second cognition metric is distinct from the first cognition metric and has a second false positive rate that is lower than the first false positive rate.

16. A system of testing a subject for impairment, comprising:
a measurement apparatus to measure the subject's gaze position;
a display;
one or more processors;
memory, the memory storing one or more programs, the one or more programs comprising instructions to:
present an object to the subject;
while presenting the object to the subject, measure, using the measurement apparatus, the gaze positions of the respective eye of the subject;
generating, using the measured gaze positions of the respective eye, a plurality of values of a gaze metric;
generate a cognition metric for the subject based on one or more of the group consisting of:
a measure of a skewness of a distribution of the plurality of values of the gaze metric; and
a measure of kurtosis of the distribution of the plurality of values of the gaze metric;
determine whether the cognition metric is indicative of cognitive impairment; and
generate a report, based at least in part on the determination of whether the cognition metric is indicative of cognitive impairment, indicating the presence or absence of cognitive impairment.

17. The system of claim 16, wherein a respective value of the plurality of values of the gaze metric corresponds to a difference between a measured gaze position and a position of the object.

18. The system of claim 16, wherein a respective value of the plurality of values of the gaze metric corresponds to a rate of change of a measured gaze position.

19. A non-transitory computer readable storage medium storing one or more programs, the one or more programs comprising instructions that when executed by one or more processors of a computer system operatively coupled to a display and a measurement apparatus to measure a subject's gaze position, cause a system that includes the computer system, display and measurement apparatus to:
present an object to the subject;
while presenting the object to the subject, measure, using the measurement apparatus, the gaze positions of the respective eye of the subject;
generating, using the measured gaze positions of the respective eye, a plurality of values of a gaze metric;
generate a cognition metric for the subject based on one or more of the group consisting of:
a measure of a skewness of a distribution of the plurality of values of the gaze metric; and
a measure of kurtosis of the distribution of the plurality of values of the gaze metric;
determine whether the cognition metric is indicative of cognitive impairment; and
generate a report, based at least in part on the determination of whether the cognition metric is indicative of cognitive impairment, indicating the presence or absence of cognitive impairment.

20. The non-transitory computer readable storage medium of claim 19, wherein a respective value of the plurality of values of the gaze metric corresponds to a difference between a measured gaze position and a position of the object.
